(19) United States
(12) Patent Application Publication
(10) Pub. No.: US 2008/0286393 A1
(43) Pub. Date: Nov. 20, 2008

(54) Method for the enrichment of the polyphenolic contents from unripe fruit rinds of Terminalia belerica and uses thereof

(76) Inventor: Muhammed Majeed, Piscataway, NJ (US)

Correspondence Address: SABINSA CORPORATION, 70 ETHEL ROAD WEST, UNIT 6, PISCATAWAY, NJ, 08854, US

(21) Appl. No.: 11/750,350

(22) Filed: May 18, 2007

(51) Int. Cl. A61K 36/00; C07C 39/04; A61Q 5/00; A61Q 19/00

(52) U.S. Cl. 424/769; 568/717

(57) ABSTRACT

An improved method for concentrating/enriching the polyphenolic contents from the dried, unripe fruit rinds of Terminalia belerica is disclosed. The present invention also discloses a light colored, enriched/concentrated (85%-100%) polyphenolic extract from the dried, unripe fruit rinds of Terminalia belerica which, by virtue of its excellent skin lightening, anti-aging, anti-inflammatory, anti-oxidant, UV protective, anti-microbial, sebum control, anti-acne and safety properties shows tremendous nutra-cosmetic potential as topical/oral “skin care” and “hair care” formulations. Additionally, the oral nutra-cosmetic formulations containing the light colored, enriched/concentrated (85%-100%), polyphenolic extract from the dried, unripe fruit rinds of Terminalia belerica, in view of their excellent 5-alpha-reductase activity, provide a natural therapeutic alternative to the problems of Benign prostatic hyperplasia (BPH), prostate cancer, and prostatitis.

### FIELD OF THE INVENTION

The present invention in general relates to the extraction of polyphenols from fruits. In particular, the invention relates to (i) an improved method to concentrate/enrich the polyphenolic contents of dried, unripe fruit rinds of Terminalia belerica; (ii) a light colored, concentrated polyphenolic extract from the dried, unripe fruits of Terminalia belerica; and (iii) the potential applications of the light colored, enriched/concentrated (85%-100%) polyphenolic extract from the dried, unripe fruit rinds of Terminalia belerica as topical and oral nutra-cosmetic formulations for “skin care” and “hair care”.

### BACKGROUND OF THE INVENTION

“Terminalia belerica” or “Terminalia bellirica” is a deciduous tree. The tree belongs to the Family Combretaceae and is distributed throughout in the deciduous forests of India. The tree is referred to as “Belliric Myrobalan” in English, as “Vibhitaki”, “Karashaphala” and “Kalidruma” in Sanskrit and as “Bahera” in Hindi. The most commonly used part of the plant is the fruit. The fruits are green and inflated when young and yellowish and ribbed when mature. The principal constituents of the fruit include β-sitisterol, gallic acid, ellagic acid, ethyl gallate, galloyl glucose and chebulagic acid. Terminalia belerica is a preventative and rejuvenating supplement alone or in the “triphala” formula along with equal proportions of Emblica Officinalis and Terminalia Chebula.

The numerous applications of Terminalia belerica fruit have been documented as prior art references. A review of the important prior art literature is presented herein below.

Fruit extracts of Terminalia belerica in association with other fruit extracts from different plants have been shown to regulate skin pigmentation in WO9624327 titled “Compositions having depigmenting activity, and uses thereof” to Hanna Raja on Aug. 15, 1996.

The antiviral effects of the fruit peel of Terminalia belerica Roxb along with other plant based extracts has been documented in EP0568001 titled “Antiviral agent containing crude drug” to Hozumi Toyoharu on Nov. 3, 1993.

The efficacy of the fruit extracts of Terminalia belerica in association with other herbal extract in the treatment of HIV infections in patients is documented in WO2005030232 titled “Herbal compositions for effective treatment of aids, preparation thereof and method for treatment of AIDS patients” to Ayare Shambabu on Apr. 7, 2005.

A peroxylipid inhibitor, comprising fruit extracts of Emblica officinalis, Terminalia chebula Retz. or Terminalia belerica with a hydrophilic solvent is discussed in JP4069343, titled “Peroxylipid formation inhibitor composed of plant extract” to Kojima Hiroyuki on Mar. 4, 1992.

The fruit extracts of Terminalia belerica in combination with other chosen herbal extracts are used as herbal cough formulations in U.S. Publication No. 20030228383 titled “Herbal cough formulations and process for the preparation thereof” to Doshi, Madhukat Mansukhlal on Dec. 11, 2003.

Synergistic, anti-allergic herbal compositions comprising fruit extracts of Terminalia belerica with other herbal extracts have been disclosed in U.S. Pat. No. 6,730,332, titled “Herbal composition having anti-allergic properties and a process for the preparation thereof” to Agarwal, Ravindra Kumar on May 5, 2004.

The efficacy of Terminalia belerica fruit extract in controlling Helicobacter pylori infections has been discussed in U.S. Pat. No. 6,187,313, titled “Composition and method for treating and preventing Helicobacter-pylori-associated stomach gastritis, ulcers and cancer” to Segelman, Alvin Burton on Feb. 13, 2001.

The manufacture and use of herbal compositions comprising combinations of Melia azadirachta and other extracts including Terminalia belerica for the treatment of skin conditions and/or the promotion of general good health, and in particular, for the treatment of psoriasis, eczema and lichen planus in human and non-human animals is discussed in U.S. Pat. No. 5,693,327, titled “Herbal compositions” to Shah, Eladevi on Dec. 2, 1997.

The drug “vibhitaki” occurring the fruit rind extract of Terminalia belerica characterized as being ‘bitter, acrid, astringent, laxative, germicidal and antipyretic’ and its therapeutic applications in cough, tuberculosis, eye diseases, dyspepsia, diarrhoea, dysentery, inflammation of the small intestine, biliousness, flatulence, liver disease, leprosy, blood purification, voice promotion and hair growth promotion have been documented by Sivarajan V. V in 1994. Indian Journal of Pharmacy 1963:297.

The in-vitro antioxidant activity of “Triphala”, an equi-proportional mixture of Emblica officinalis, Terminalia chebula and Terminalia belerica has been documented in the publication titled “In vitro antioxidant studies and free radical reactions of triphala, an ayurvedic formulation and its constituents” authored by Naik G H, Priyadarsini K I, Bhagirathi R G, Mishra B, Mishra K P, Banavalikar M M, Mohan H in Phytother Res. 2005 July; 19(7):582-6.

The publication titled “Immunomodulatory activity of triphala on neutrophil functions.” authored by Srikumar R, Jeya Parthasarathy N, Sheela Devi R. in Biol Pharm Bull. 2005 August; 28(8):1398-403 teaches that the oral administration of Triphala appears to stimulate the neutrophil functions in the immunized rats and stress induced suppression in the neutrophil functions were significantly prevented by Triphala.

The publication titled “Protective effect of fruit extracts of Emblica officinalis (Gaertn). & Terminalia belerica (Roxb.) in experimental myocardial necrosis in rats” authored by Tariq M, Hussain S J, Asif M, Jahan M. in Indian J Exp Biol. 1977 June; 15(6):485-6 is yet another example of a documented use of Terminalia belerica (Roxb.)

The hypo-lipidemic effect of “triphala” on experimentally induced hypercholesterimic rats has been documented by Saravanan S, Srikumar R, Manikandan S, Jeya Parthasarathy N, Sheela Devi R. in Yakugaku Zasshi. 2007 February; 127(2):385-8.

The use of Terminalia belerica in ancient Egyptian folk medicine has been documented by El-Mekkawy S, et al. Chemical & Pharmaceutical Bulletin 1995; 43: 641-648.

The anti-asthmatic, anti-spasmodic, expectorant and anti-tussive effects of “vibhitaki” (T. belerica) has been elucidated by Trivedi et. al., in the Journal of Research in Ayurveda and Siddha (1979), 3, 1-8) through an open clinical study in 93 patients suffering from respiratory conditions.

With a plethora of prior art on the multifarious applications of Terminalia belerica fruit extracts in combinations with other herbal extracts, the present inventors sought to exclusively study in detail, the polyphenolic contents of the Terminalia belerica fruit.

U.S. Pat. No. 5,932,623 titled “Process for the production of fruit polyphenols from unripe Rosaceae fruit” to Tanabe, Masayuki on Aug. 3, 1999 discloses the extraction of polyphenolic contents from fruits like apples and peaches by adsorbing the polyphenols onto an adsorbent after evaporating the alcohols from the fruit extract, removal of solids from the fruit extract and dissolving the fruit extract in hexane or chloroform. However, the use of hexane or chloroform may affect the quantity and quality of the yield of polyphenols from the fruits.

The present inventors thus felt an immediate need for an alternative, effective method to concentrate/enrich the polyphenolic contents from the unripe fruit rinds of Terminalia belerica that would facilitate a more detailed analysis of the useful properties of the said polyphenolic extract.

It is thus the principle object of the present invention to develop an improved method to concentrate/enrich the polyphenolic contents from the unripe fruit rinds of Terminalia belerica.

It is another object of the present invention to recover a light-colored, concentrated/enriched polyphenolic extract from the unripe fruit rinds of Terminalia belerica which is most desired in the cosmetics industry.

It is yet another object of the present invention to evaluate the light-colored, concentrated/enriched polyphenolic extract from the fruit rinds of Terminalia belerica for its skin lightening, anti-aging, anti-inflammatory, anti-oxidant, UV protective, anti-microbial, sebum control, anti-acne and safety properties.

Further, it is another object of the present invention to develop oral and topical nutra-cosmetic formulations containing the light-colored, concentrated/enriched polyphenolic extracts from the unripe fruit rinds of Terminalia belerica for “skin care” and “hair care”.

The present invention fulfills all these objectives and provides further related advantages that serve as useful addendums to the existing knowledge on Terminalia belerica.

### SUMMARY OF THE INVENTION

An improved method for concentrating/enriching the polyphenolic contents from the unripe fruit rinds of Terminalia belerica is disclosed. The improved method comprises of subjecting the powdered, unripe fruit rinds of Terminalia belerica to Super Critical Carbon-dioxide Extraction method to remove the non-polar and semi-polar fractions that may interfere with the yield and quality of the polyphenolic contents, prior to extraction steps involving the polar solvents and non-polar solvents as known in the art. The present invention also discloses a light colored, concentrated/enriched (85%-100%) polyphenolic extract from the dried, unripe fruit rinds of Terminalia belerica, properties and applications thereof.

The advantageous features of the present invention include:
a) An improved method to enrich/concentrate the polyphenolic contents from the dried, unripe fruit rinds of Terminalia belerica.
b) A concentrated/enriched (85%-100%) polyphenolic extract from the dried, unripe fruit rinds of Terminalia belerica which is most desired in the cosmetic industry due to its light color when compared to polyphenolic extracts from other plant sources.
c) A light-colored, concentrated/enriched (85%-100%) polyphenolic extract from the fruit rinds of Terminalia belerica that exhibits skin lightening, anti-aging, anti-inflammatory, anti-oxidant, UV protective, anti-microbial, sebum control and anti-acne properties.
d) Topical and oral nutra-cosmetic formulations containing a light-colored, concentrated/enriched (85%-100%) polyphenolic extracts from the unripe fruit rinds of Terminalia belerica that serve to provide excellent “skin care” by (i) improving the skin tone/complexion and (ii) preventing/reducing the pathologies associated with the phenomenon of skin aging including, but not limited to diminished rate of turn-over of skin cells; reduced skin microcirculation; weak mechanical resistance of the skin due to flattening of the dermal-epidermal junction; formation of superficial or deep/coarse wrinkles, crevices and bumps on the skin; hyper-pigmentation of the skin including the appearance of age spots and freckles; Loss of skin firmness and elasticity leading to sallowness, sagging, blotching, dark under-eye circles and puffy eyes; enlarged skin pores; abnormal desquamation or exfoliation or the skin cells; visible dead skin caused due to flaking and scaling.
e) Topical and oral nutra-cosmetic formulations containing light-colored, enriched/concentrated (85%-100%) polyphenolic extract from the dried, unripe fruit rinds of Terminalia belerica that provide excellent overall “hair care” and in particular, a solution to the problems of hair fall.
f) Additionally, the oral nutra-cosmetic formulations containing the light colored, enriched/concentrated (85%-100%) polyphenolic extract of Terminalia belerica of the present invention, in view of their 5-alpha-reductase activity, provide a natural therapeutic alternative to the problems of Benign prostatic hyperplasia (BPH), prostate cancer, and prostatitis.
g) Topical and oral nutra-cosmetic formulations containing the light-colored concentrated/enriched polyphenolic extract from the unripe fruit rinds of Terminalia belerica, and which exhibit an enhanced safety profile for application on the skin and mucous membranes.

Other features and advantages of the present invention will become apparent from the following more detailed description, taken in conjunction with the accompanying figures, which illustrate, by way of example, the principle of the invention.

### DETAILED DESCRIPTION OF THE FIGURES

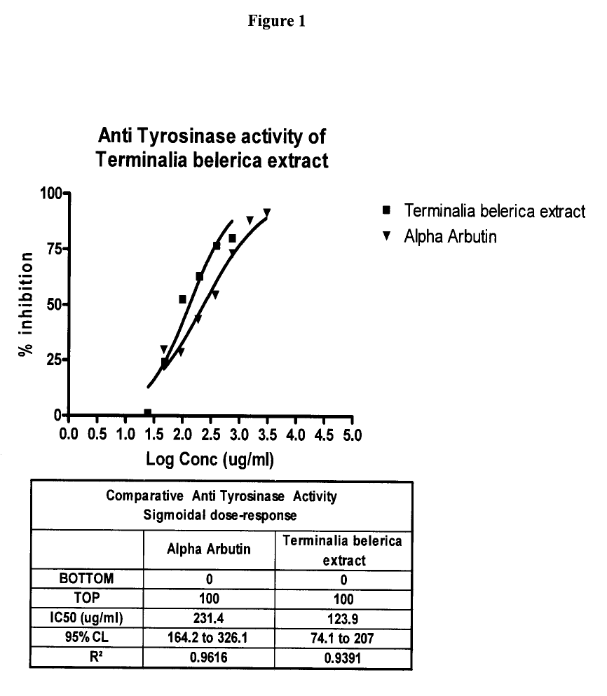

FIG. 1 shows a comparative sigmoidal dose response curve of the anti-tyrosinase activity of α-Arbutin and the light-colored, enriched/concentrated (85%-100%) polyphenolic extract from the dried, unripe fruit rinds of Terminalia belerica.

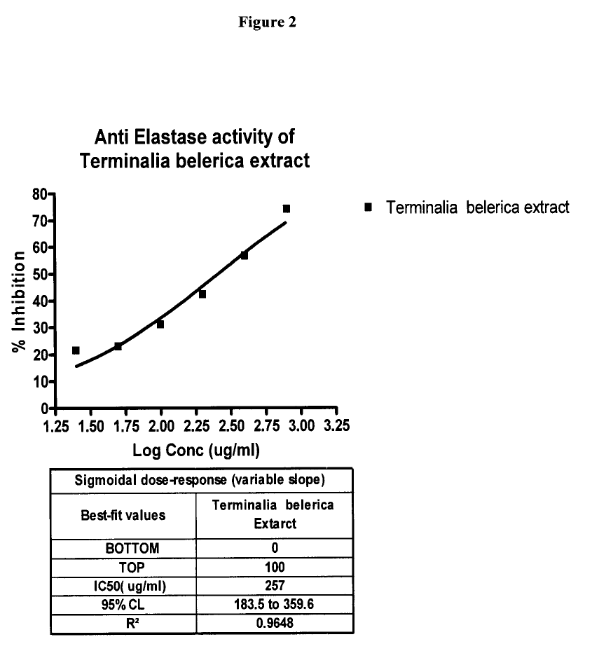

FIG. 2 shows a sigmoidal dose response curve of the anti-elastase activity of the light-colored enriched/concentrated (85%-100%) polyphenolic extract from the dried, unripe fruit rinds of Terminalia belerica.

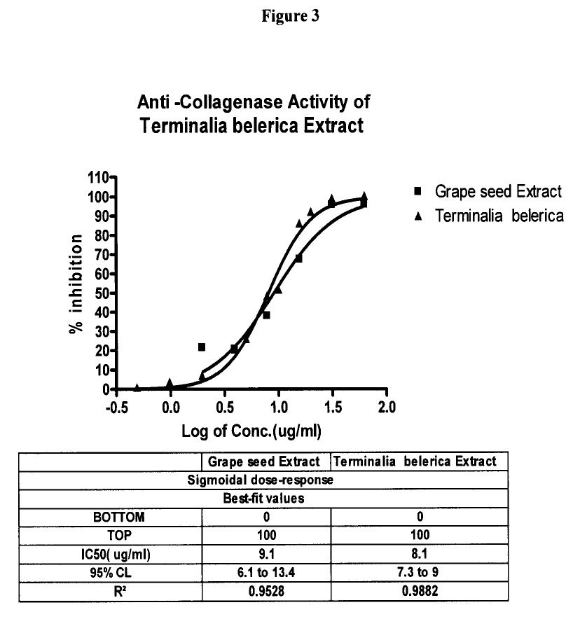

FIG. 3 shows a comparative sigmoidal dose response curve of the anti-collagenase activity of grape seed extract and the light-colored, enriched/concentrated (85%-100%) polyphenolic extract from the dried, unripe fruit rinds of Terminalia belerica.

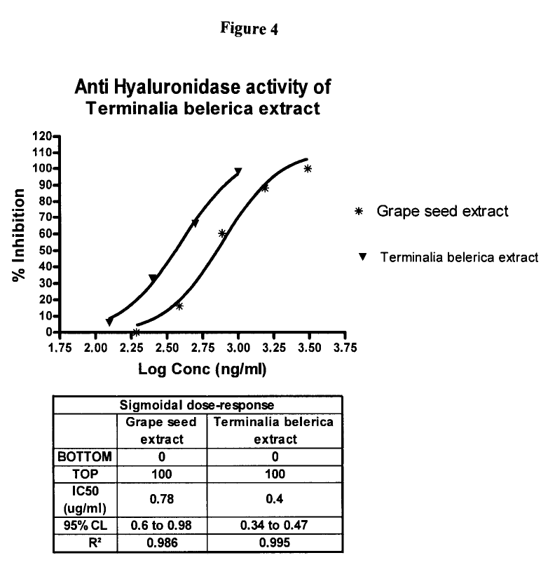

FIG. 4 shows a comparative sigmoidal dose response curve of the anti-hyaluronidase activity of grape seed extract and the light-colored, enriched/concentrated (85%-100%) polyphenolic extract from the dried, unripe fruit rinds of Terminalia belerica.

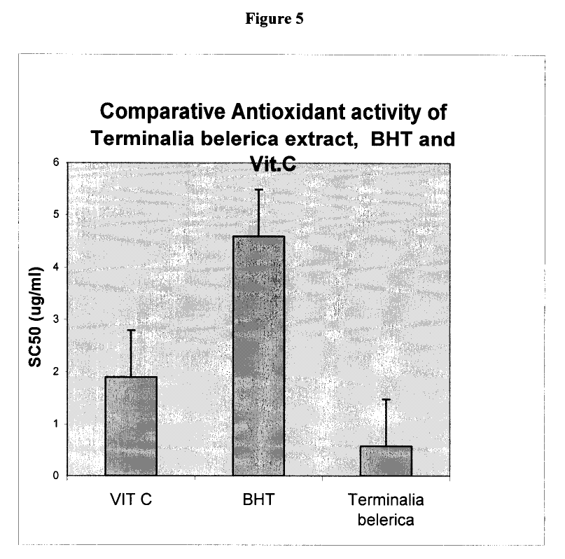

FIG. 5 shows the graphical representation of the comparative anti-oxidant activity of Vitamin C, Butylated hydroxyl toluene (BHT) and the light-colored, enriched/concentrated (85%-100%) polyphenolic extract from the dried, unripe fruit rinds of Terminalia belerica.

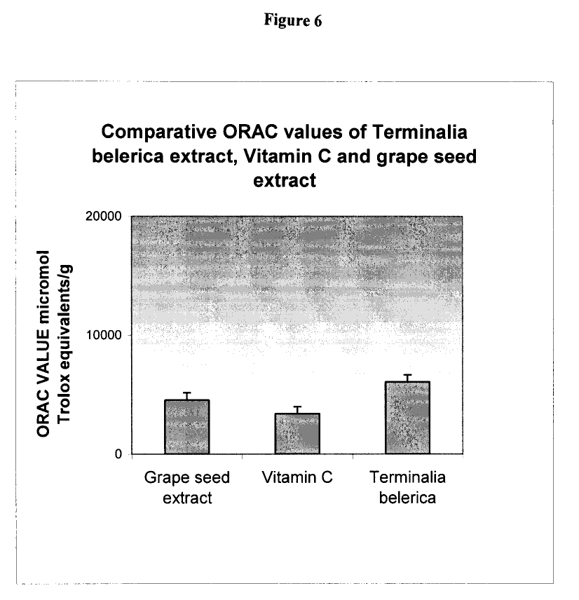

FIG. 6 shows the graphical representation of the comparative the oxygen radical scavenging activity (ORAC) of Grape seed extract, vitamin C and the light-colored, enriched/concentrated (85%-100%) polyphenolic extract from the dried, unripe fruit rinds of Terminalia belerica.

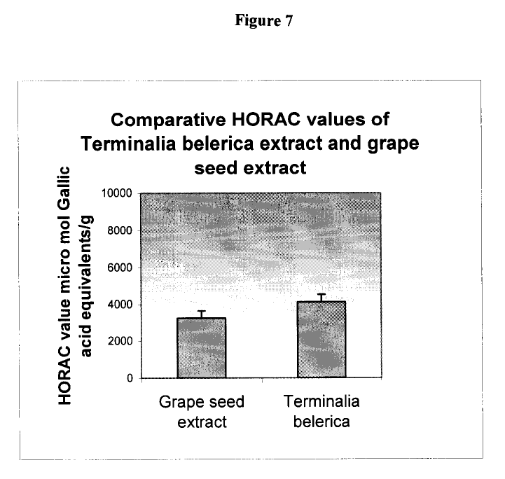

FIG. 7 shows the graphical representation of the comparative the hydroxyl radical averting capacity (HORAC) of Grape seed extract and the light-colored, enriched/concentrated (85%-100%) polyphenolic extract from the dried, unripe fruit rinds of Terminalia belerica.

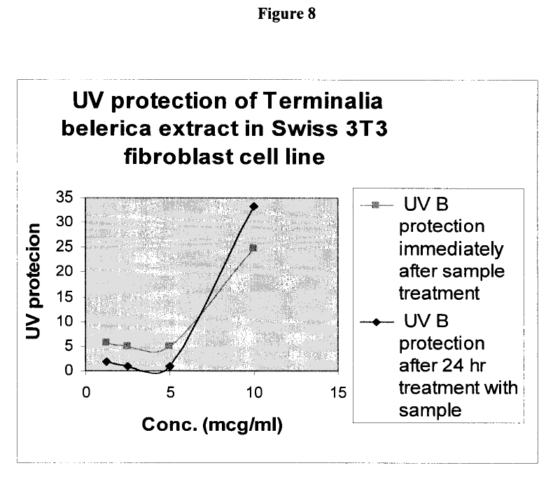

FIG. 8 shows the UV B protection conferred to Swiss 3T3 fibroblast cell lines by the light-colored, enriched/concentrated (85%-100%) polyphenolic extract from the dried, unripe fruit rinds of Terminalia belerica (i) immediately after treatment (ii) 24 hours after treatment

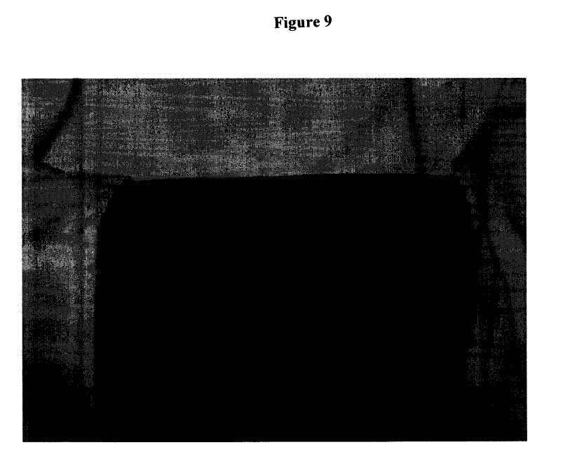

FIG. 9 shows a picture of the UV B protection conferred to the skin of a human subject by varying concentrations of the light-colored, enriched/concentrated (85%-100%) polyphenolic extract from the dried, unripe fruit rinds of Terminalia belerica.

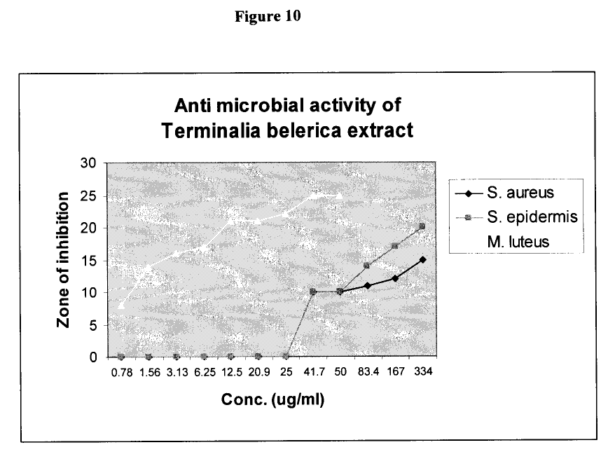

FIG. 10 shows a graphical representation of the antimicrobial Activity of the light-colored, enriched/concentrated (85%-100%) polyphenolic extract from the dried, unripe fruit rinds of Terminalia belerica studied by Kirby-Bauer's Disc Diffusion method against Staphylococcus aureus NCIM2079, Staphylococcus epidermidis NCIM2493, and Micrococcus luteus NCIM 2169 strains.

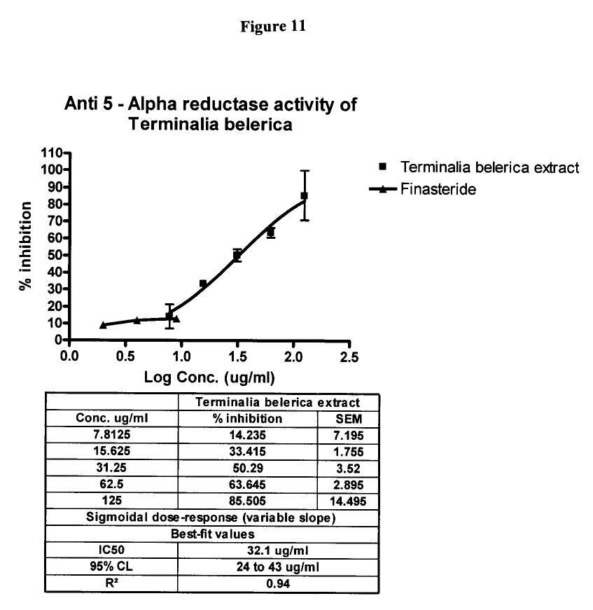

FIG. 11 shows a graphical representation of the anti-5-alpha-reductase activity of the increasing concentrations of the light-colored, enriched/concentrated (85%-100%) polyphenolic extract from the dried, unripe fruit rinds of Terminalia belerica.

### DETAILED DESCRIPTION OF THE MOST PREFERRED EMBODIMENT

In the most preferred embodiment, the present invention discloses an improved method to concentrate/enrich the polyphenolic contents from the dried, unripe fruit rinds of Terminalia belerica. Further, the said improved method ensures a very light colored powder of the enriched/concentrated (85%-100%) polyphenolic extract from the dried, unripe fruit rinds of Terminalia belerica which is most desired in the nutra-cosmetic industry.

The improved method involves subjecting the powdered, unripe fruit rinds of Terminalia belerica to Super Critical Carbon dioxide extraction process to remove the non-polar and semi-polar components that may interfere with the concentration/enrichment of the polyphenolic contents, prior to extraction steps using polar solvents and non-polar solvents well known in the art. The Super Critical Carbon dioxide technique carried out in the present invention is explained in detail herein below.

The shade dried, unripe fruit rinds (seeds removed) of Terminalia belerica are powdered and subjected to Super Critical Carbon dioxide Extraction using an ‘entrainer’ selected from a group of monohydric and dihydric alcohols (C4 to C6 carbons). In a preferred embodiment, the entrainer percentage is between 5 to 15%. More preferably, the entrainer percentage is between 8 to 10%. A pressure range between 250 to 300 bars is applied during the extraction process. More preferably, a pressure of 285 bars is applied during the procedure. In the most preferred embodiment, the non-polar and semi-polar components are removed from the fruit rinds by precipitating them at about 80 to 100 bars. A temperature range of 35° to 65° C. is maintained during the Super Critical Carbon dioxide Extraction process. More preferably, the temperature range is 45° to 55° C. In the most preferred embodiment, the Super Critical Carbon dioxide Extraction is carried out at 50° C. The extraction procedure lasts for about 3 to about 4 hours. Following the removal of the non-polar and semi-polar components by the Super Critical Carbon dioxide Extraction technique, the spent fruit rind powder is subjected to normal extraction process well known in the art, the steps of which are elucidated herein below.
a) Extracting thrice, the spent fruit rind powder, with polar organic solvents including, but not limited to water, ethanol, methanol, isopropyl alcohol, butanol, mixture of water and C1-C4 alcohols, under hot conditions at 45-90° C. for 1-6 hrs. More preferably, the extraction is carried out at a temperature of 70-80° C. for 3-4 hrs.
b) The extractives obtained in step (a) are filtered and the filtrate is concentrated under reduced pressure at 45° C.-60° C. to obtain an extract containing 50-60% of polyphenols.
c) The extract obtained in step (b) is further enriched with a non-polar solvent including but not limited to toluene, Ethylacetate, chlorinated solvents by fractionation for 1-4 hrs.
d) The non-polar fractions obtained in step (c) are then concentrated and dried to obtain a cream color powder containing 85%-100% polyphenols.
e) The assay of the concentrated polyphenols obtained in step (d) is performed by the Folin-Denis method.

In another most preferred embodiment, the light-colored, enriched/concentrated (85%-100%) polyphenolic extract from the dried, unripe fruit rinds of Terminalia belerica as mentioned herein above exhibits excellent skin lightening, anti-aging, anti-inflammatory, anti-oxidant, UV protective, anti-microbial, sebum control, anti-acne and safety properties. The aforesaid properties of the enriched/concentrated (85%-100%) polyphenolic extract from the dried, unripe fruit rinds of Terminalia belerica are set forth as specific examples herein below.

### Example I

### Skin lightening (Inhibition of Tyrosinase Activity)

The skin lightening effect of the light-colored, enriched/concentrated (85%-100%) polyphenolic extract from the dried, unripe fruit rinds of Terminalia belerica was studied using the method proposed in the publication titled “Inhibitory Activity of Flavone derivative isolated from Sophora flavescens” in Bull. Korean. chem. Soc. (2001), 22 (1), 97-99. The activity of the mushroom tyrosinase enzyme was analyzed in the presence of the light-colored, enriched/concentrated (85%-100%) polyphenolic extract from the dried, unripe fruit rinds of Terminalia belerica. Dopachrome produced is determined by reading the absorbance at 492 nm in a microplate reader (BMG Fluostar Optima Microplate Reader). Inhibition results were calculated in Graph pad prism software and expressed as IC₅₀ values for the concentration of the light-colored, enriched/concentrated (85%-100%) polyphenolic extract from the dried, unripe fruit rinds of Terminalia belerica required for 50% of the enzyme to be inhibited. FIG. 1 shows that the light-colored, enriched/concentrated (85%-100%) polyphenolic extract from the dried, unripe fruit rinds of Terminalia belerica had a superior anti-tyrosinase activity in comparison to Arbutin, a well known skin lightener as measured by the IC₅₀ value of 123.9 μg/ml.

### Example II

### Anti-Elastase Activity

The anti-elastase assays were done using the EnzChek elastase assay kit. The test is based on the principle that the florescence exhibited by DQ elastin from soluble bovine neck ligament (substrate) labeled with BODIPY FL dye is quenched in the presence of an elastase inhibitor. The reduction in fluorescence intensity is measured in a microplate reader/Fluostar Optima (emission at 485 nm and excitation at 520 nm.) Inhibition results are calculated in Graph pad prism software and expressed as IC₅₀ values, the concentration of the sample required for 50% of the enzyme to be inhibited. Aliquots of the enzyme solution (100 μl of 0.5 U/ml of elastase from pig pancreas) and different concentrations of the light-colored, enriched/concentrated (85%-100%) polyphenolic extract from the dried, unripe fruit rinds of Terminalia belerica in double distilled Water (50 μl) were pre-incubated in a microplate for 10 minutes. After pre-incubation, 50 μl of the substrate DQ elastin (25 μg/ml) (EnzChek) was added and the fluorescence intensity was measured after 30 minutes. FIG. 2 shows increased inhibition of elastase activity by increasing concentrations of the light-colored, enriched/concentrated (85%-100%) polyphenolic extract from the dried, unripe fruit rinds of Terminalia belerica. The IC₅₀ value for the anti-elastase activity of enriched/concentrated (85%-100%) polyphenolic extract from the dried, unripe fruit rinds of Terminalia belerica. was 257 μg/ml.

### Example III

### Anti-Collagenase Activity

The anti-collagenase assays were done using the EnzChek Collagenase assay kit. The test is based on the principle that, the fluorescence exhibited by DQ gelatin from pig skin (substrate) labeled with BODIPY FL dye is quenched in the presence of a collagenase inhibitor. The fluorescence intensity is measured in a microplate reader (emission at 485 nm and excitation at 520 nm) and the results are expressed as IC50 values, the concentration of the inhibitor that causes 50% of enzyme inhibition when compared to the activity of the enzyme in absence of inhibitor. 80 μl of different concentrations of the light-colored, enriched/concentrated (85%-100%) polyphenolic extract from the dried, unripe fruit rinds of Terminalia belerica in double distilled water was pre-incubated in a black well plate for 10 minutes with 20 μl of the BODIPY FL dye labeled DQ gelatin substrate. 100 μl aliquots of the collagenase enzyme solution was added and the fluorescence intensity was measured after 30 minutes (at Em: 485 nm and Ex: 520 nm). FIG. 3 shows that the anti-collagenase activity of the light-colored, enriched/concentrated (85%-100%) polyphenolic extract from the dried, unripe fruit rinds of Terminalia belerica is comparable in efficacy to the grape seed extract known for its anti-collagenase activity. The IC₅₀ value for the anti-collagenase activity of the light-colored, enriched/concentrated (85%-100%) polyphenolic extract from the dried, unripe fruit rinds of Terminalia belerica was 8.1 μg/ml.

### Example IV

### Anti-Hyaluronidase Activity

Hyaluronic acid substrate (0.3%) was made in 300 mM sodium phosphate buffer pH 5.35. Hyaluronidase (10 U/ml) was made in 20 mM Sodium phosphate Buffer pH 7.00. 1 ml enzyme and 20 μl of the various concentrations of the light-colored, enriched/concentrated (85%-100%) polyphenolic extract from the dried, unripe fruit rinds of Terminalia belerica in DMSO (1%) were pre-incubated at 37° C. for 10 min. Then 1 ml of the substrate was added and the reaction mixture was incubated for 45 min at 37° C. To 0.5 ml of the reaction mix was added to 2.5 ml of cetyl pyrimidine chloride (1%). The absorbance of undigested hyaluronic acid was read at 600 nm. Controls were kept with the vehicle (DMSO) and the substrate. Blanks for each conc. of the sample were kept without substrate. Calculation: The results expressed as IC₅₀ values (the concentration of the inhibitor which there is 50% inhibition of enzyme when compared to the activity of the enzyme in absence of inhibitor) as shown in FIG. 4 indicate that the anti-hyaluronidase activity of the light-colored, enriched/concentrated (85%-100%) polyphenolic extract from the dried, unripe fruit rinds of Terminalia belerica is superior to that of grape seed extract. The IC₅₀ value for anti-hyaluronidase activity of the light-colored, enriched/concentrated (85%-100%) polyphenolic extract from the dried, unripe fruit rinds of Terminalia belerica was 0.4 μg/ml.

### Example V

### Anti-Oxidant Activity

The antioxidant activity of the light-colored, enriched/concentrated (85%-100%) polyphenolic extract from the dried, unripe fruit rinds of Terminalia belerica was assayed by diphenylpicrylhydrazyl (DPPH), Oxygen Radical Absorption (or Absorbance) Capacity (ORAC) and the hydroxyl radical Absorption (or Absorbance) capacity (HORAC) studies, as per the validated procedures documented in Cosmetics and toiletries. Cosmeceuticals: Jang D I, Lee B J, Jeon C O, Jo N S, Park J H, Cho S Y, Lee H, Koh J S. 1998, J. Agric. Food Chem. (2001), 49, 4619-4626 and J. Agric. Food. Chem. (2002), 50, 2772-2777.)

FIG. 5 shows that the scavenging activity of the light-colored, enriched/concentrated (85%-100%) polyphenolic extract from the dried, unripe fruit rinds of Terminalia belerica extract was superior to Vitamin C and Butylated hydroxyl toluene (BHT) as indicated by the lower SC₅₀ value (0.57 μg/ml) of the concentrated/enriched (85%-100%) polyphenolic extract from the unripe fruit rinds of Terminalia belerica extract in comparison to that of Vitamin C (1.9 μg/ml) and BHT (4.6 μg/ml).

FIG. 6 shows that the Oxygen Radical Absorption (or Absorbance) Capacity (ORAC) of the light-colored, enriched/concentrated (85%-100%) polyphenolic extract from the dried, unripe fruit rinds of Terminalia belerica was found to be superior to Vitamin C and grape seed extract as indicated by the respective ORAC values. The ORAC value of the light-colored, enriched/concentrated (85%-100%) polyphenolic extract from the dried, unripe fruit rinds of Terminalia belerica was 6237±640 μMol trolox equivalents/gram while the ORAC value of Vitamin C was 3400 μMol trolox equivalents/gram and that of grape seed extract was 4528 μMol trolox equivalents/gram.

FIG. 7 shows that the Hydroxyl Radical Absorption (or Absorbance) Capacity (HORAC) of the light-colored, enriched/concentrated (85%-100%) polyphenolic extract from the dried, unripe fruit rinds of Terminalia belerica (4117573 μMol Gallic acid equivalents/g) was higher than that of grape seed extract (3254 μMol Gallic acid equivalents/g).

### Example VI

### UV Protective Activity

In-Vitro Testing

The in-vitro study is based on the ability of the light-colored, enriched/concentrated (85%-100%) polyphenolic extract from the dried, unripe fruit rinds of Terminalia belerica to reduce photo damage caused by UVB to Swiss 3T3 fibroblast cell line. The cell viability is studied as the marker for UV protective function. (J Am Acad Dermatol September 2003) Calculation of UV protection was based on the difference in % cell viability in the presence and absence of the extract (non toxic concentrations) during exposure. FIG. 8 shows that Swiss 3T3 Fibroblast cell lines are protected from UV B photo damage when treated with the light-colored, enriched/concentrated (85%-100%) polyphenolic extract from the dried, unripe fruit rinds of Terminalia belerica immediately following UV B exposure. Further, FIG. 8 also shows that a 24 hour pre-treatment of Swiss 3T3 Fibroblast cell lines with the light-colored, enriched/concentrated (85%-100%) polyphenolic extract from the dried, unripe fruit rinds of Terminalia belerica offered protection against photo damage by UV B. UV B bulbs providing 100 μwatt/cm2 irradiance i.e., 0.14 J/cm2 irradiation dose were used as the irradiation source in the experiments.

In Vivo Testing

The human skin was exposed to UV B irradiation in the presence and absence of enriched/concentrated (85%-100%) polyphenolic extract from the dried, unripe fruit rinds of Terminalia belerica. The relative erythema and resulting pigmentation were studied. A UVB lamp having a wavelength of 270-310 nm was used for the studies. Different percentage concentrations of the light-colored, enriched/concentrated (85%-100%) polyphenolic extract from the dried, unripe fruit rinds of Terminalia belerica in water were applied on definite areas of the skin (2 cm diameter) of the right leg of the subject and exposed to UV B (3.1 W) for five minutes at a distance of 12 cm from the irradiation source. The control area (2 cm in diameter) were applied with plain water and exposed to UV B (3.1 W) for five minutes at a distance of 12 cm from the irradiation source. The test and control areas were checked for erythema. The minimum time for erythema development was noted.

Table I shows that a dose dependant UV B protection was conferred on the human skin by the light-colored, enriched/concentrated (85%-100%) polyphenolic extract from the dried, unripe fruit rinds of Terminalia belerica. The highest SPF value (calculated by dividing the time taken for minimal erythema formation in the presence of the test compound by the time taken for minimal erythema formation in the absence of the test compound) was obtained at a 5% concentration of the light-colored, enriched/concentrated (85%-100%) polyphenolic extract from the dried, unripe fruit rinds of Terminalia belerica.

The UV B induced pigmentation on the subject after 14 days was scored to study the efficacy of the light-colored, enriched/concentrated (85%-100%) polyphenolic extract from the dried, unripe fruit rinds of Terminalia belerica. Table I and FIG. 9 indicate a marked reduction in pigmentation in the presence of the light-colored, enriched/concentrated (85%-100%) polyphenolic extract from the dried, unripe fruit rinds of Terminalia belerica. There was reduction in pigmentation at all the concentrations tested. However, the best visible reduction in pigmentation was obtained at 5% concentration of the light-colored, enriched/concentrated (85%-100%) polyphenolic extract from the dried, unripe fruit rinds of Terminalia belerica.

TABLE I

| | Time taken | | | |
|---|---|---|---|---|
| | for the | | Skin | |
| | appearance of | | colour | Skin colour |
| Concentration | erythema | | Before | 14 days after |
| (%) | (Secs) | SPF | exposure | exposure |
| Control | 210 | | + | ++++++++++ |
| 0.5 | 1000 | 4.8 | | ++++++ |
| 1.0 | 1200 | 5.7 | | +++++ |
| 2.0 | 3600 | 17.1 | | +++++ |
| 3.0 | 4500 | 21.4 | | ++ |
| 5.0 | 5460 | 26 | | + |

### Example VII

### Anti-Microbial Activity

Antimicrobial Activity of the light-colored, enriched/concentrated (85%-100%) polyphenolic extract from the dried, unripe fruit rinds of Terminalia belerica was studied by the Kirby-Bauer Disc Diffusion method against Staphylococcus aureus NCIM2079, Staphylococcus epidermidis NCIM2493, and Micrococcus luteus NCIM 2169 strains. The bacterial inoculum was prepared in nutrient broth. Sterile Mueller Hinton agar plates were surface seeded with diluted 16-18 hour old bacterial culture to form a lawn culture. Sterile filter paper discs impregnated with two fold serial dilutions of the light-colored, enriched/concentrated (85%-100%) polyphenolic extract from the dried, unripe fruit rinds of Terminalia belerica were equidistantly placed on the surface of the inoculated plates. Filter paper disks impregnated with antibiotics were used as controls. The inoculated plates were incubated at 35°-37° C. for 18-24 hours. the light-colored, enriched/concentrated (85%-100%) polyphenolic extract from the dried, unripe fruit rinds of Terminalia belerica showed superior activity against Staphylococcus aureus NCIM2079 and Staphylococcus epidermidis NCIM2493 strains. The MIC values for both the strains of microorganisms were 41.7 mg/ml. This extract also showed good activity against Micrococcus luteus NCIM 2169 strains with an MIC value of 0.78 mg/ml. (Table II, FIG. 10)

TABLE I

| | Zone of inhibition (mm) | | |
|---|---|---|---|
| | Staphylococcus | Staphylococcus | Micrococcus |
| | aureus | epidermidis | luteus |
| Concentration (mg/ml) | (NCIM2079) | (NCIM2493) | (NCIM 2169) |
| 1.56 | — | — | 14 |
| 0.78 | — | — | 8 |
| 0.39 | — | — | Nil |
| 0.195 | — | — | Nil |
| 333.6 | 15 | 20 | — |
| 166.8 | 12 | 17 | — |
| 83.4 | 11 | 14 | — |
| 50 | — | — | 25 |
| 41.7 | 10 | 10 | — |
| 25 | — | — | 22 |
| 20.85 | Nil | Nil | — |
| 12.5 | — | — | 21 |
| 6.25 | — | — | 17 |
| 3.13 | — | — | 16 |

### Example VIII

### 5-Alpha-Reductase Activity

Activity of the 5-Alpha-reductase enzyme (from rat liver microsomes) on substrate testosterone (40 μM) was assayed in presence of cofactor NADPH (56 μM) and the inhibitor, the light-colored, enriched/concentrated (85%-100%) polyphenolic extract from the dried, unripe fruit rinds of Terminalia belerica over a time interval of 15 minutes. The absorbance was measured at 340 nm. The IC₅₀ value for the anti-5-Alpha-reductase activity of the light-colored, enriched/concentrated (85%-100%) polyphenolic extract from the dried, unripe fruit rinds of Terminalia belerica was found as 32.1 μg/ml. (FIG. 11)

### Example IX

### Safety Studies

Primary Skin Irritation Test in Rabbits

The study was designed to determine the potential of the light-colored, enriched/concentrated (85%-100%) polyphenolic extract from the dried, unripe fruit rinds of Terminalia belerica (test substance) to cause primary skin irritation in rabbits. 500 mg of the test substance was applied to intact and abraded skin of three rabbits belonging to either sexes. The sites of application were carefully observed and the reaction was evaluated at 24 and 72 hours in accordance with the Draize's method. No erythema or edema of skin was observed in rabbits after application of the test substance and the light-colored, enriched/concentrated (85%-100%) polyphenolic extract from the dried, unripe fruit rinds of Terminalia belerica did not cause any skin irritation in rabbits. The primary skin irritation score was 0.00.

Mucous Membrane Irritation in Rabbits

The study was designed to determine the potential of the light-colored, enriched/concentrated (85%-100%) polyphenolic extract from the dried, unripe fruit rinds of Terminalia belerica (test substance) to cause mucous membrane irritation in rabbits. 100 mg of the test substance was instilled on the everted lower lid of the right eye of each test animal. The left eye of each animal was untreated and served as control. The treated eyes of animals were flushed with water after 24 hrs. Conjunctival, iridial and corneal irritation were evaluated at 24, 48, 72 and 96 hrs post treatment and scored in accordance with Draize's method. the light-colored, enriched/concentrated (85%-100%) polyphenolic extract from the dried, unripe fruit rinds of Terminalia belerica did not cause any mucous membrane irritation in rabbits.

The examples set for the herein above clearly illustrate tremendous nutra-cosmetic potential for the light-colored, enriched (85%-100%) polyphenolic extract from the fruit rinds of Terminalia belerica as topical and oral “skin care” and “hair care” formulations.

In yet another most preferred embodiment, topical and oral nutra-cosmetic formulations containing the light-colored, concentrated/enriched (85%-100%) polyphenolic extracts from the unripe fruit rinds of Terminalia belerica that serve to provide excellent “skin care” by (i) improving the skin tone/complexion and (ii) preventing/reducing the pathologies associated with the phenomenon of skin aging including, but not limited to diminished rate of turn-over of skin cells; reduced skin microcirculation; weak mechanical resistance of the skin due to flattening of the dermal-epidermal junction; formation of superficial or deep/coarse wrinkles, crevices and bumps on the skin; hyper-pigmentation of the skin including the appearance of age spots and freckles; Loss of skin firmness and elasticity leading to sallowness, sagging, blotching, dark under-eye circles and puffy eyes; enlarged skin pores; abnormal desquamation or exfoliation or the skin cells; visible dead skin caused due to flaking and scaling are disclosed.

In yet another preferred embodiment, topical and oral nutra-cosmetic formulations containing the light colored, enriched/concentrated (85%-100%), polyphenolic extract of Terminalia belerica that provide excellent overall hair care, in particular, a solution to the problems of hair loss is disclosed.

In an additional embodiment, the oral nutra-cosmetic formulations containing the light colored, enriched/concentrated (85%-100%), polyphenolic extract of Terminalia belerica in view of their 5-alpha-reductase activity provide a natural therapeutic alternative to the problems of Benign prostatic hyperplasia (BPH), prostate cancer, and prostatitis.

While the invention has been described with reference to a preferred embodiment, it is to be clearly understood by those skilled in the art that the invention is not limited thereto. Rather, the scope of the invention is to be interpreted only in conjunction with the appended claims.

What is claimed is:

1. In the art for the recovery of polyphenolic contents from fruits, an improvement in the methods of concentrating/enriching the polyphenolic contents from dried, unripe fruit rinds of Terminalia belerica, said improvement consisting essentially of subjecting the fruit rinds to Super Critical Carbon dioxide Extraction process to remove the non-polar and semi-polar components that may interfere with the concentration/enrichment of the polyphenols prior to the other steps involved in the said recovery process.

2. The improvement in the methods of concentrating/enriching the polyphenolic contents from dried, unripe fruit rinds of Terminalia belerica according to claim 1, wherein the powdered, unripe fruit rinds (seeds removed) of Terminalia belerica are subjected to Super Critical Carbon Dioxide Extraction using an ‘entrainer’ selected from the group of monohydric and dihydric alcohols.

3. The improvement in the methods of concentrating/enriching the polyphenolic contents from dried, unripe fruit rinds of Terminalia belerica according to claim 2, wherein the monohydric alcohols are selected from the group consisting of methyl alcohol, ethyl alcohol, isopropyl alcohol and n-butyl alcohol.

4. The improvement in the methods of concentrating/enriching the polyphenolic contents from dried, unripe fruit rinds of Terminalia belerica according to claim 2, wherein the dihydric alcohols are selected from the group consisting of propylene glycol, butylenes glycol and hexanediol.

5. The improvement in the methods of concentrating/enriching the polyphenolic contents from dried, unripe fruit rinds of Terminalia belerica according to claim 1, wherein the Super Critical Carbon dioxide Extraction is done at a pressure of 250 to 300 bars.

6. The improvement in the methods of concentrating/enriching the polyphenolic contents from dried, unripe fruit rinds of Terminalia belerica according to claim 5, wherein the Super Critical Carbon dioxide Extraction is done at a pressure of 285 bars.

7. The improvement in the methods of concentrating/enriching the polyphenolic contents from dried, unripe fruit rinds of Terminalia belerica according to claim 5, wherein the non-polar and semi-polar components are removed from the said fruit rinds by precipitating them at about 80 to 100 bars.

8. The improvement in the methods of concentrating/enriching the polyphenolic contents from dried, unripe fruit rinds Terminalia belerica according to claim 1, wherein the Super Critical Carbon dioxide Extraction is carried out at a temperature range between 45° to 55° C.

9. The improvement in the methods of concentrating/enriching the polyphenolic contents from dried, unripe fruit rinds of Terminalia belerica according to claim 8, wherein the Super Critical Carbon dioxide Extraction is carried out at a temperature of 50° C.

10. A light colored, enriched/concentrated (85%-100%) polyphenolic extract recovered from the dried, unripe fruit rinds of Terminalia belerica by an improved method comprising the steps of:
a) Subjecting the powdered, unripe fruit rinds of Terminalia belerica to Super Critical Carbon dioxide Extraction process to remove the non-polar and semi-polar components that may interfere with the concentration/enrichment of the polyphenolic contents.
b) Extracting thrice, the spent fruit rind powder resulting from step (a) with polar organic solvents under hot conditions.
c) Filtering the extractives obtained in the step (b) and concentrating the filtrate under reduced pressure at 45° C.-60° C. to obtain an extract containing 50-60% of polyphenols.
d) Enriching the extract obtained in the step (c) with non-polar solvents.
e) Concentrating the non-polar fractions obtained in the step (d) and drying the concentrate to obtain a cream color powder containing 85%-100% polyphenols.
f) Assaying the concentrated polyphenols obtained in step (e).

11. The light colored, enriched/concentrated (85%-100%) polyphenolic extract according to claim 10, wherein the said polyphenolic extract exhibits excellent skin lightening, anti-aging, anti-inflammatory, anti-oxidant, UV protective, anti-microbial, sebum control, anti-acne and safety properties.

12. The polyphenolic extract according to claim 11, wherein the said extract exhibits anti-tyrosinase activity with an IC₅₀ value of 123.9 μg/ml.

13. The polyphenolic extract according to claim 11, wherein the said extract exhibits anti-elastase activity with an IC₅₀ value of 257 μg/ml.

14. The polyphenolic extract according to claim 11, wherein the said extract exhibits anti-collagenase activity with an IC₅₀ value of 8.1 μg/ml.

15. The polyphenolic extract according to claim 11, wherein the said extract exhibits anti-hyaluronidase activity with an IC₅₀ value of 0.4 μg/ml.

16. The polyphenolic extract according to claim 11, wherein the said extract exhibits anti-oxidant activity with SC₅₀ value of 0.57 μg/ml for diphenylpicrylhydrazyl (DPPH) radicals.

17. The polyphenolic extract according to claim 11, wherein the said extract exhibits anti-oxidant activity with an Oxygen Radical Absorption (or Absorbance) Capacity (ORAC) of 6237±640 μMol trolox equivalents/gram.

18. The polyphenolic extract according to claim 11, wherein the said extract exhibits anti-oxidant activity with a Hydroxyl Radical Absorption (or Absorbance) Capacity (HORAC) of 4117±573 μMol Gallic acid equivalents/gram.

19. The polyphenolic extract according to claim 11, wherein the said extract exhibits in-vitro UV B protective function.

20. The polyphenolic extract according to claim 11, wherein the said extract exhibits in vivo UV B protective function.

21. The polyphenolic extract according to claim 11, wherein the said extract exhibits superior anti-microbial activity as measured by the Kirby-Bauer Disc Diffusion method.

22. The polyphenolic extract according to claim 21, wherein the said extract exhibits an MIC (Minimum inhibitory Concentration) value of 41.7 mg/ml for the strains of Staphylococcus aureus NCIM2079.

23. The polyphenolic extract according to claim 21, wherein the said extract exhibits an MIC (Minimum inhibitory Concentration) value of 41.7 mg/ml for the strains of Staphylococcus epidermidis NCIM2493.

24. The polyphenolic extract according to claim 21, wherein the said extract exhibits an MIC (Minimum inhibitory Concentration) value of 0.78 mg/ml for the strains of Micrococcus luteus NCIM 2169.

25. The polyphenolic extract according to claim 11, wherein the said extract exhibits anti-5-alpha-reductase activity with an IC 50 value of 32.1 μg/ml.

26. The polyphenolic extract according to claim 10, wherein the said extract shows tremendous nutra-cosmetic potential as oral and topical “skin care” and “hair care” formulations.

27. The polyphenolic extract according to claim 26, wherein the said extract provides excellent “skin care” by improving skin tone and skin complexion.

28. The polyphenolic extract according to claim 26, wherein the said extract prevents and reduces characteristic pathologies associated with the phenomenon of skin ageing.

29. The polyphenolic extract according to claim 26, wherein the said extract provides excellent overall “hair care”.

30. The polyphenolic extract according to claim 26, wherein the said extract prevents hair fall.

31. The polyphenolic extract according to claim 10, wherein the said extract exhibits an enhanced safety profile for application on the skin and mucous membranes.

32. The polyphenolic extract as in any one of claims 10, 11 and 25, in which the said polyphenolic extract provides a natural therapeutic alternative to the problems of Benign prostatic hyperplasia (BPH), prostate cancer, and prostatitis.
